(19) United States
(12) Patent Application Publication
(10) Pub. No.: US 2014/0236268 A1
(43) Pub. Date: Aug. 21, 2014

(54) FAR INFRARED BODY STRAP THAT SOOTHES PAIN

(71) Applicants: Brent Allen Safer, (US); Ronald Leslie Ferguson, (US)

(72) Inventor: Brent Allen Safer, Elizabeth, CO (US)

(73) Assignees: Ronald Leslie Ferguson, Elizabeth, CO (US); Brent Allen Safer, Elizabeth, CO (US)

(21) Appl. No.: 13/768,755

(22) Filed: Feb. 15, 2013

(51) Int. Cl. A61N 5/06

(52) U.S. Cl. CPC A61N 5/0613
USPC 607/90

(57) ABSTRACT

The present invention is formed by a body strap, far infrared releasers that can be increased or decreased, electricity connectors and a controller. The far infrared releasers on the body strap are connected serially for use by the electricity connectors. Connection assemblies are then disposed at body parts where pain needs to be soothed and then snapped together after positions of the connection assemblies have been adjusted properly in order to facilitate soothing pain to body parts, vitalizes blood circulation and activates cells. In addition, the far infrared releasers can be increased, decreased or replaced on demand.

### BACKGROUND OF THE INVENTION

a) Field of the Invention

The present invention relates to a far infrared body strap that soothes pain, and more particularly to a far infrared body strap that soothes pain, wherein far infrared releasers can be increased or decreased on demand and can be tightly attached at every body part where pain needs to be soothed, so as to achieve the effect of soothing pain.

b) Description of the Prior Art

As change of lifestyle and struggle for existence of a modern man, life stress, work stress and environmental stress increase rapidly. Therefore, most of the modern men who live busily will have uncomfortableness like soreness or tightness at their shoulders, necks, waists, backs or lower bodies. Some people will go to a massage shop for a session of stress relieving; whereas, most people will purchase far infrared related products for partial soothing.

That the abovementioned far infrared is able to achieve the functions of soothing stress, vitalizing blood circulation and activating cells is primarily due to that far infrared can stimulate a human body, is able to boost up human energy, and result in the effects of resonance absorption and treatment to a human body. Furthermore, far infrared that gets deeper into a human body will cause vibration to atoms and molecules, forming a heat reaction through the resonance absorption to increase hypodermic temperature, expand capillaries, promote blood circulation, remove completely obstacles such as bruises that impede metabolism, revive tissues and facilitate growth of enzymes. After this complete metabolism, wastes and harmful accumulation that have been left inside a human body will be expelled out of the body along with sweat glands and water.

Therefore, there are all kinds of massage products or soothing products on the markets, claiming to be equipped with the far infrared effects. However, in using these products, it is unable to increase or decrease far infrared releasers depending upon a need of every body part or to wear the far infrared releasers on the body serially to adjust a limit of stress relieving. Thus, far infrared stress relieving products that fit all parts of a human body should be purchased, which is inconvenient in usage and costs more money relatively to have these products.

Accordingly, how to solve the abovementioned problems and shortcomings of the prior arts is to be researched and developed for improvement by the present inventor and related vendors.

### SUMMARY OF THE INVENTION

The primary object of the present invention is to provide a far infrared body strap that soothes pain, with that the far infrared body strap can be emplaced at every body part where pain needs to be soothed, thereby achieving the effect of stress relieving.

To achieve the abovementioned object, the present invention discloses a far infrared body strap that soothes pain, including a body strap with an elastic effect, the body strap being provided with plural bags and two ends of the body strap being provided respectively with a connection assembly, with the connection assemblies being combined together; plural far infrared releasers, each far infrared releaser being provided respectively with a positive electrode connection member and a negative electrode connection member, with one far infrared releaser being installed in one bag; plural electricity connectors, each electricity connector being provided with a second plug that is inserted into the positive electrode connection member and a first plug that is inserted into the negative electrode connection member; and a controller, the controller being provided with a power receptacle that is inserted with the first or second plug, with the controller being disposed in one bag.

According to an embodiment of the present invention, each far infrared releaser is provided with plural LED (Light Emitting Diode) light sources to release far infrared.

According to an embodiment of the present invention, the body strap is formed with plural openings that correspond respectively to each bag and provide for the LED light sources to release far infrared outward.

According to an embodiment of the present invention, the body strap is provided with a washable pad at an opening surface that is in contact with a human body.

According to an embodiment of the present invention, the connection assemblies include a male joint and a female joint that is snapped with the male joint.

According to an embodiment of the present invention, the far infrared body strap further includes an extension fastener set that is connected with the connection assemblies to increase a limit of snapping.

According to an embodiment of the present invention, the extension fastener set includes a male fastener that is engaged with the female joint and a female fastener that is engaged with the male joint.

A second object of the present invention is to provide a far infrared body strap that soothes pain, wherein by using the extension fastener set, a limit of fixation can be increased for a user with a wider body, allowing the far infrared body strap to be more tightly attached to the body.

A third object of the present invention is to provide a far infrared body strap that soothes pain, wherein through replacing the far infrared releasers, the far infrared releasers can be increased, decreased or replaced for maintenance at will according to a practical need of the user.

A fourth object of the present invention is to provide a far infrared body strap that soothes pain, wherein the far infrared releasers are electrically connected in a serial arrangement, which facilitates the user to replace and operate by himself or herself.

A fifth object of the present invention is to provide a far infrared body strap that soothes pain, wherein the method of using the body strap enables the user to wear the body strap with a good wrapping ability to meet the need of every body part, in a more diversified way.

A sixth object of the present invention is to provide a far infrared body strap that soothes pain, wherein the far infrared releasers can be attached on a human body or be used with an assistance of a bracket, on human body positions where the body strap is unable to be applied conveniently.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a three-dimensional schematic view of a preferred embodiment of the present invention.

FIG. 2 shows a schematic view of an operation of installing far infrared releasers, according to the present invention.

FIG. 3 shows a schematic view of a usage state of the preferred embodiment of the present invention.

FIG. 4 shows a schematic view of a usage state of replacing a pad of the present invention.

FIG. 5 shows a schematic view of a usage state of applying an extension fastener set of the present invention.

### DETAILED DESCRIPTION OF THE PREFERRED EMBODIMENTS

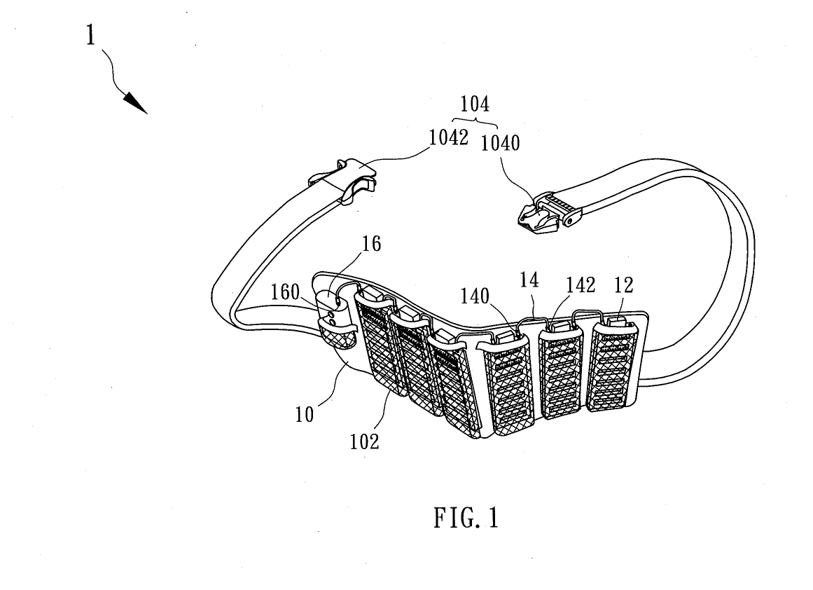

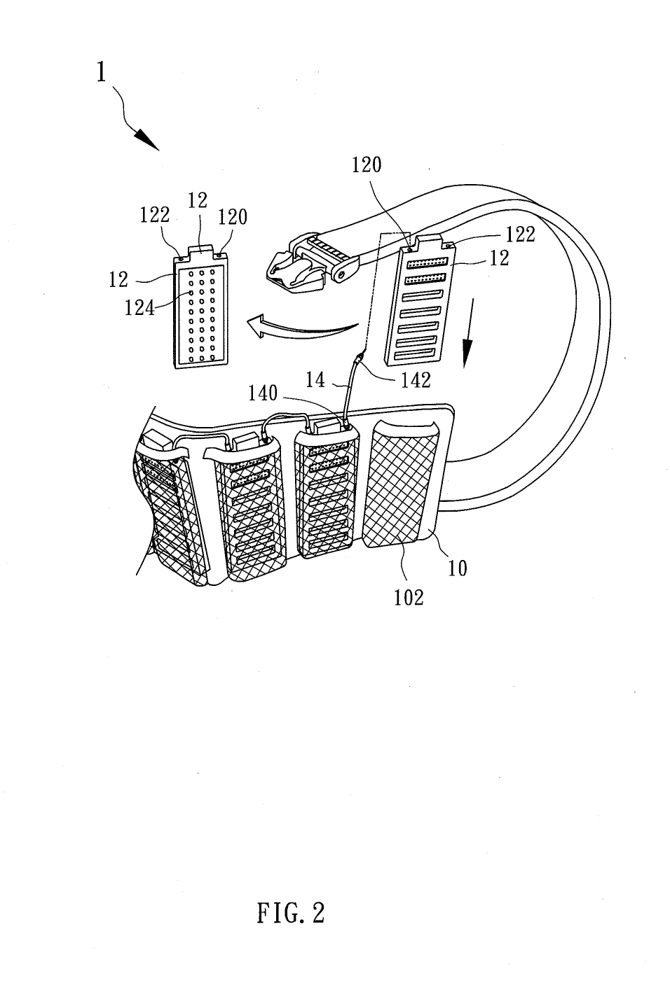

Referring to FIG. 1 and FIG. 2, it shows a three-dimensional schematic view of a preferred embodiment of the present invention and a schematic view of an operation of installing far infrared releasers, according to the present invention. As shown in the drawings, the present invention provides a far infrared body strap that soothes pain 1, comprising a body strap 10, the body strap 10 being provided with plural bags 102 and two ends of the body strap 10 being provided respectively with a connection assembly 104, with the connection assemblies 104 being combined together; plural far infrared releasers 12, one far infrared releaser 12 being installed in one bag 102 and each far infrared releaser 12 being provided with a positive electrode connection member 120 and a negative electrode connection member 122; plural electricity connectors 14, each electricity connector 14 being provided respectively with a second plug 142 that is inserted into the positive electrode connection member 120 and a first plug 140 that is inserted into the negative electrode connection member 122; and a controller 16, the controller 16 being provided with a power receptacle 160 that is inserted with the first or second plug 140, 142, with the controller 16 being disposed in one bag 102.

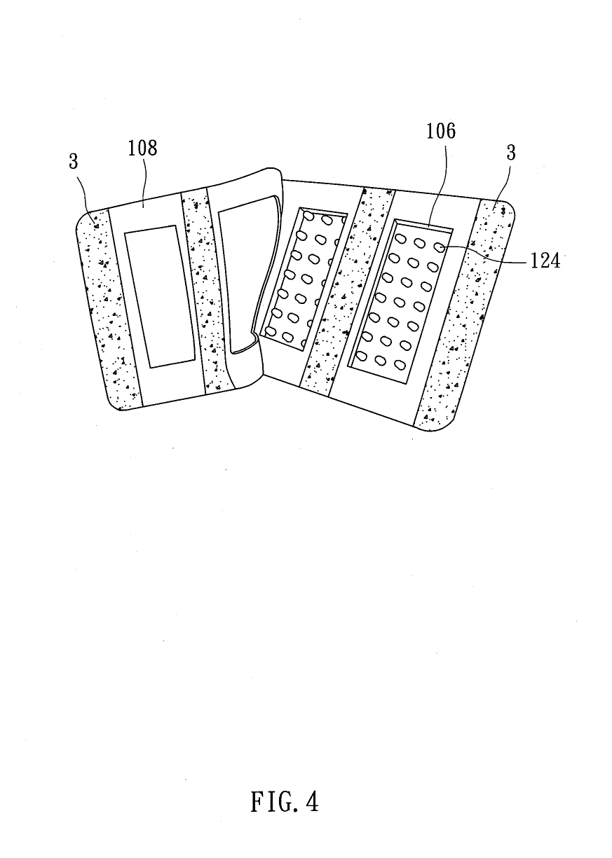

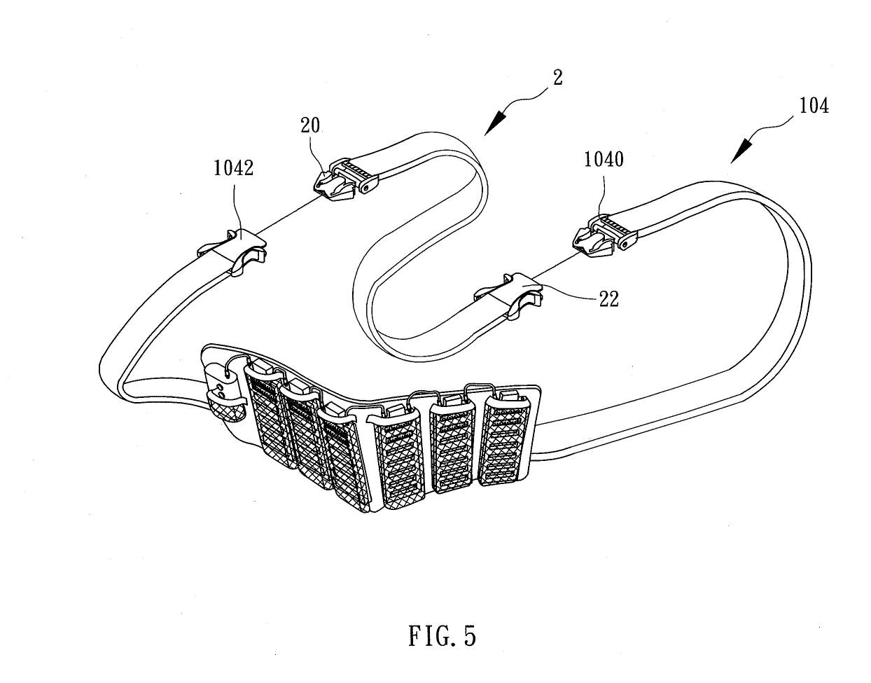

Each abovementioned far infrared releaser 12 is provided with plural LED light sources 124 to release far infrared and each LED light source 124 is emplaced respectively at an opening 106 (as shown in FIG. 4) on the body strap 10, with one opening 106 corresponding to one bag 102, so as to allow the LED light sources 124 to release far infrared outward. The LED light sources 124 can be replaced with any specification that fits a need, as required. Furthermore, the body strap 10 is provided with a pad 108 at a surface that is in contact with a human body (as shown in FIG. 4). The abovementioned connection assemblies 104 include a male joint 1040 and a female joint 1042 that is snapped with the male joint 1040. For a user with a wider body, the present invention is provided with an extension fastener set 2 to increase a limit of use of the far infrared body strap that soothes pain 1 (as shown in FIG. 5). The extension fastener set 2 is connected with the connection assemblies 104, and includes a male snap 20 that is engaged with the female joint 1042 and a female snap 22 that is engaged with the male joint 1040.

As shown in FIG. 2, the user can increase or decrease the far infrared releasers 12 according to a practical need. After the number of far infrared releasers 12 has been determined, the first plug 140 and the second plug 142 of each electricity connector 14 are inserted respectively into the negative electrode connection member 122 and the positive electrode connection member 120, allowing the far infrared releasers 12 to be electrically connected in a serial arrangement. Next, the far infrared releasers 12 are connected to the power receptacle 160 on the controller 16 (as shown in FIG. 1) and this accomplishes the entire assembly. Following that, the far infrared body strap that soothes pain 1 is hung on a back or tied at a body part where pain needs to be soothes, such as a shoulder, neck, waist, back or lower part that the user feels uncomfortable, like soreness or tightness.

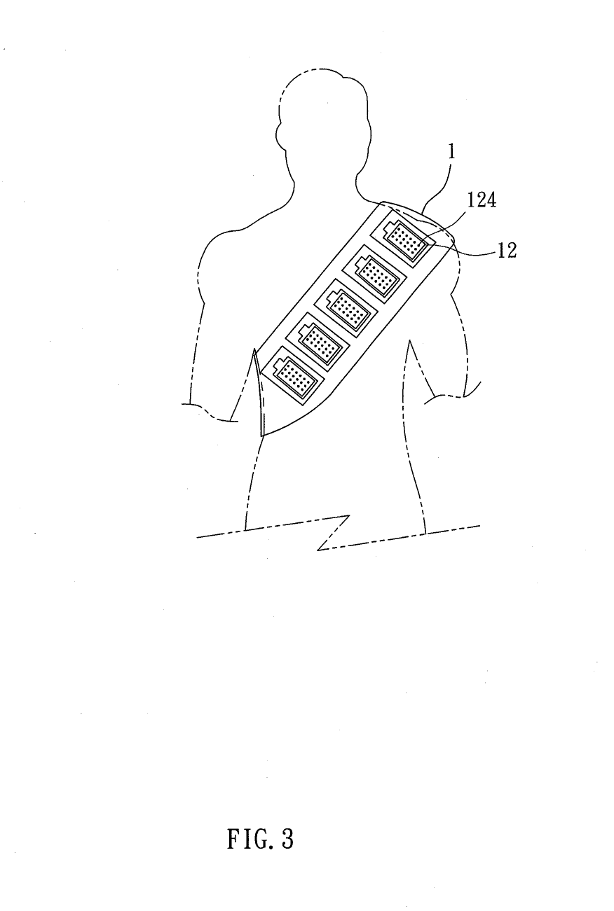

As shown in FIG. 3 and in association with FIGS. 1 and 2, the far infrared body strap that soothes pain 1 is hung at a waist as an example.

The user first attached tightly the far infrared releasers 12 at a human body toward the direction that the LED light sources 124 release far infrared, followed by wrapping the connection assemblies 104 around the body and adjusting the length of the connection assemblies 104 properly for comfortableness and for being effectively and tightly fixed at the body. Next, the male joint 1040 and the female joint 1042 are snapped together (the abovementioned connection assemblies 104 are drawn in FIG. 1), which accomplishes wearing the far infrared body strap 1. Finally, the controller 16 is activated to commence with stress relieving.

As each part of a human body is different in shape, for a human body position where the body strap is unable to be applied conveniently, the far infrared releasers 12 can be attached individually on the human body or be used with an assistance of bracket (e.g., on a face).

On the other hand, referring to FIG. 4, it shows a schematic view of a usage state of replacing the pad, according to the present invention. The surface of the far infrared body strap that soothes pain 1 that is in contact with the human body is added with the pad 108. As the surface that is in contact with the human body can be stained by sweat or other factors, the pad 108 that can be installed and dismantled easily is added to facilitate cleaning and replacing, thereby maintaining sanitary. The pad 108 can be adhered by a hook and loop fastener 3 or fixed by a male and female fastener; in this embodiment, the hook and loop fastener 3 is used.

Referring to FIG. 5, it shows a schematic view of a usage state of applying the extension fastener set 2 of the present invention. The present invention is primarily added by the extension fastener set 2 that is connected to the connection assemblies 104 to increase the limit of snapping. The extension fastener set 2 includes the male snap 20 that is engaged with the female joint 1042 and the female snap 22 that is engaged with the male joint 1040. Therefore, for the user of a wider body, the limit of fixation can be increased, so that the user can have a better soothing effect, without feeling that the body is tied up firmly.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. A far infrared body strap that soothes pain, comprising:
a body strap with an elastic effect, wherein the body strap being provided with plural bags and two ends of the body strap being provided respectively with a connection assembly, with the connection assemblies being combined together;
plural far infrared releasers, wherein each far infrared releaser being provided respectively with a positive electrode connection member and a negative electrode connection member, with one far infrared releaser being installed in one bag;
plural electricity connectors, wherein each electricity connector being provided respectively with a second plug that is inserted into the positive electrode connection member and a first plug that is inserted into the negative electrode connection member; and
a controller, wherein the controller being provided with a power receptacle that is inserted with the first or second plug, with the controller being disposed in one bag.

2. The far infrared body strap that soothes pain, according to claim 1, wherein each far infrared releaser is provided with plural LED (Light Emitting Diode) light sources to release far infrared.

3. The far infrared body strap that soothes pain, according to claim 1, wherein the body strap is provided with a pad at an opening surface that is in contact with a human body.

4. The far infrared body strap that soothes pain, according to claim 1, wherein the body strap connection assemblies include a male joint and a female joint that is snapped with the male joint.

5. The far infrared body strap that soothes pain, according to claim 4, further including an extension fastener set that is connected with the connection assemblies in order to increase a limit of snapping.

6. The far infrared body strap that soothes pain, according to claim 5, wherein the extension fastener set includes a male snap that is engaged with the female joint and a female snap that is engaged with the male joint.

7. The far infrared body strap that soothes pain, according to claim 1, wherein the bags and the far infrared releasers are increased, decreased or replaced for maintenance on demand.

8. The far infrared body strap that soothes pain, according to claim 2, wherein the LED light sources are replaced with any specification as required.

9. The far infrared body strap that soothes pain, according to claim 1, wherein the far infrared releasers are attached on a human body position where the body strap is unable to be applied conveniently.

10. The far infrared body strap that soothes pain, according to claim 1, wherein the far infrared releasers are used with an assistance of a bracket on a human body position where the body strap is unable to be applied conveniently.
